(19) United States
(12) Patent Application Publication
(10) Pub. No.: US 2023/0399363 A1
(43) Pub. Date: Dec. 14, 2023

(54) BACULOVIRUS EXPRESSION VECTOR

(71) Applicant: Intervet Inc., Madison, NJ (US)

(72) Inventor: Amaya Serrano Garcia, BOXMEER (NL)

(73) Assignee: Intervet Inc., Madison, NJ (US)

(21) Appl. No.: 18/249,419

(22) PCT Filed: Oct. 21, 2021
(86) PCT No.: PCT/EP2021/079156

§ 371 Date: Apr. 18, 2023

(30) Foreign Application Priority Data
Oct. 22, 2020 (EP) 20203373.4

(51) Int. Cl. C07K 14/005; C12N 15/86; A61K 39/135

(52) U.S. Cl. CPC C07K 14/005; C12N 15/86; A61K 39/135; C12N 2710/14043; C12N 2770/32122; C12N 2770/32151; C12N 2770/32134; A61K 2039/5258

(57) ABSTRACT

The invention concerns a baculovirus expression vector for recombinantly expressing an FMDV capsid precursor protein under control of a promoter, the expression vector comprising a nucleic acid sequence encoding the FMDV capsid precursor protein and the translational enhancers Syn2 land p10UTR. The invention further relates to a host cell comprising the baculovirus expression vector, a method of producing FMDV virus-like particles (VLPs), and a method of producing a vaccine.

The invention concerns a baculovirus expression vector for recombinantly expressing a Foot-and-mouth disease virus (FMDV) capsid precursor protein under control of a promoter, the expression vector comprising a nucleic acid sequence encoding the FMDV capsid precursor protein and a translational enhancer. The invention further relates to a host cell comprising the baculovirus expression vector, a method of producing FMDV virus-like particles (VLPs), and a method of producing a vaccine.

### BACKGROUND OF THE INVENTION

Foot-and-mouth disease (FMD) is a highly contagious, acute viral disease of cloven-hoofed, domesticated and wild animals. It is classified as a transboundary animal disease by the Food and Agriculture Organisation of the United Nations (FAO). It is also a notifiable disease. Foot-and-mouth disease is endemic in large parts of Africa, South America, The Middle East and Asia and is, globally, the most economically important infectious disease of livestock, affecting cattle, pigs, sheep, goats and other artiodactyl species like buffalo and deer. FMD was once distributed worldwide but has been eradicated in some regions, including North America and Western Europe. In endemic countries, FMD places economic constraints on the international livestock trade and can be easily reintroduced into disease-free areas unless strict precautions are in place. FMD impacts on the whole livestock industry with loss of income for local farmers.

Current vaccines are made of inactivated virus. Before the virus is inactivated, live FMD virus is produced in high containment facilities, limiting FMD vaccine production. Effective vaccination against FMD requires the presence of intact FMDV capsids rather than the capsid building blocks that have been proven to be insufficiently immunogenic (Doel and Chong, 1982, Archives of Virology). The inactivated FMD viruses are fragile structures that at acidic pH or at elevated temperatures easily fall apart in the capsid building blocks. Hence, a cold chain is required to deliver effective FMD vaccines to livestock keepers.

A new vaccine technology for commercial FMD vaccines that can overcome many of the drawbacks of the current inactivated virus vaccines is needed.

The virus-like particle (VLP) technology is currently considered one of the few technologies with the potential to be a viable alternative to conventional inactivated vaccines. The benefits of the VLP technology as compared to the current technology are for example higher product stability, greater flexibility in production location (low-containment production), and quicker responses to outbreaks of new strains. VLP-based vaccines are typically designed as marker vaccines which relieves the necessity of implementing production steps to remove non-structural proteins.

The FMDV genome encodes a single open reading frame (ORF) that produces a precursor polyprotein that is processed into twelve mature viral proteins, FIG. 1A (from: Balinda et al. Virology Journal 2010, 7:199). The P1 polyprotein intermediate is comprised of four capsid structural proteins, VP1-VP4, sited immediately upstream of the 2A protein which causes non-proteolytic separation of the P1 and P2 polyproteins during translation to release P1-2A from P2. The P1-2A polyprotein is subsequently processed by the FMDV 3C protease into 2A, VP0 (also known as 1AB), VP3 (1C), and VP1 (1D). The VP0 protein separates into VP4 and VP2 during encapsulation. FMDV virions are formed by self-assembly from the processed virus structural proteins.

VLPs for use in VLP-based vaccines can be produced by recombinantly expressing FMDV precursor proteins in suitable host cells in analogy to the self-assembly of FMDV virions. FMDV VLP's are likewise formed by self-assembly from the processed virus structural proteins.

The thermostability and sensitivity to low pH of VLPs can be improved by the introduction of covalent links between the capsid proteins, such as cysteine bridges, or by the introduction of other rationally designed mutations (Porta et al. (2013) PLoS Pathog).

Because the initial yield of FMDV VLPs per milliliter cell culture is typically low, even when using common successful expression systems, there is a need to increase expression levels to make the VLP-based FMD vaccine a cost-effective alternative to the classic FMD vaccines currently on the market. Therefore, the baculovirus expression vector was optimized to improve the yield of VLPs and to achieve a large-scale production process that at least equals or even outperforms the conventional FMD vaccine production process in terms of antigen yield.

The baculovirus expression vector platform is currently used as one of the preferred platforms for the production of VLPs. However, the relatively low expression levels of FMDV VLPs provided by the baculovirus expression platform limits the development of a VLP-based FMD vaccine.

### SUMMARY OF THE INVENTION

In the present invention, it has surprisingly been found that expression of an FMDV capsid precursor protein via the baculovirus expression system can be enhanced by using a combination of two translational enhancers: Syn21 and P10UTR. It could surprisingly be shown that the combination of these two translational enhancers outperforms expression yields achieved by other translational enhancers used in baculovirus expression systems in the art.

Thus, in a first aspect the present invention provides a baculovirus expression vector capable of recombinantly expressing an FMDV capsid precursor protein under control of a promoter, the expression vector comprising:
(i) a nucleic acid sequence encoding the FMDV capsid precursor protein,
(ii) a translational enhancer Syn21 located within the 5′ untranslated region (UTR) of the nucleic acid sequence (i) encoding the FMDV capsid precursor protein, and
(iii) a translational enhancer P10UTR, located within the 3′ UTR of the nucleic acid sequence (i) encoding the FMDV capsid precursor protein.

In a second aspect of the invention, there is provided a host cell comprising the baculovirus expression vector of the present invention. Such a host cell can be used in vitro, in a tissue culture, the host cell typically being an immortalized cell.

In a third aspect, the invention provides a method of producing FMDV VLPs, the method comprising the steps of: infecting a host cell with the baculovirus expression vector as described herein, and harvesting VLPs produced by the host cell.

The invention further relates to the use of the baculovirus expression vector for the recombinant expression of a FMDV capsid precursor protein.

The invention further relates to a method of producing a vaccine by producing FMDV VLPs and incorporating the FMDV VLPs into a vaccine by addition of a pharmaceutically acceptable carrier.

The invention further relates to a method of protecting a subject against an infection with FMDV by expressing an FMDV capsid precursor protein from the baculovirus expression vector of the present invention in a host cell to produce a VLP, incorporating the VLP into a vaccine by addition of a pharmaceutically acceptable carrier and administering the VLP to the subject.

The invention further relates to a baculovirus expression vector according to the first aspect of the invention for use in the protection of a subject against an infection with FMDV.

The invention further relates to a baculovirus expression vector as described herein for use in the manufacture of a medicament for the protection of a subject against an infection with FMDV.

### DETAILED DESCRIPTION OF THE INVENTION

### Definition of Terms

The term “nucleic acid sequence” includes an RNA or DNA sequence. It may be single or double stranded. It may, for example, be genomic, recombinant, mRNA or cDNA.

An “expression vector” (syn. “expression construct”), is usually a plasmid or virus designed for recombinant gene expression in cells. The vector is used to introduce a specific gene into a target cell, and can commandeer the cell's mechanism for protein synthesis to produce the protein of interest (POI) encoded by the gene. In order to express the recombinant gene to produce the POI, the expression vector typically comprises at least a promotor for initiating gene expression and may further comprise one or more translational enhancers.

A “baculovirus expression vector” is an expression vector based on a baculovirus, which is used for recombinant gene expression in a host cell, such as an insect cell. Baculovirus expression systems are established in the art and are commercially available, such as the Bac-to-Bac expression system (ThermoFisher Scientific, Germany). In these baculovirus expression systems, the naturally occurring polyhedrin gene within the wild-type baculovirus genome is typically replaced with a recombinant gene or cDNA. These genes are commonly under the control of the polyhedrin or p10 baculovirus promoters.

The most common baculovirus used for gene expression is Autographa californica nucleopolyhedrovirus (AcNPV). AcNPV has a large (130 kb), circular, double-stranded DNA genome. The gene of interest (GOI) is cloned into a transfer vector containing a baculovirus promoter flanked by baculovirus DNA derived from a nonessential locus, such as the polyhedrin gene. The recombinant baculovirus containing the GOI is produced by homologous recombination in insect cells between the transfer vector and the genome of the parent virus (such as AcNPV).

A “translational enhancer” is a nucleotide sequence forming an element, which can promote translation and, thereby, increase protein production. Typically, a translational enhancer may be found in the 5′ and 3′ untranslated regions (UTRs) of mRNAs. In particular, nucleotides in the 5′-UTR immediately upstream of the initiating ATG codon of the GOI may have a profound effect on the level of translation initiation.

A virus “capsid” is commonly understood in the art as the protein shell of a virus, typically enclosing its genetic material.

A “capsid precursor protein” is a precursor of one or more structural proteins, also called capsid proteins, which take part in the formation of a virus capsid or of a building block thereof. FMDV capsid precursor proteins typically comprise the structural protein P1. Since the protein P1 is processed by the FMDV 3C protease (3Cpro) into the mature VP0, VP3, and VP1 proteins, the P1 protein may also be referred to as polyprotein or proprotein. In the context of the present invention, the FMDV capsid precursor protein typically comprises at least P1 including the proteins VP1, VP2, VP3 and VP4. Alternatively, the FMDV capsid precursor protein may comprise one or more of the proteins VP1, VP2, VP3 and VP4. The FMDV capsid precursor protein may also comprise the protein VP0 comprising the proteins VP2 and VP4. Most preferably, the FMDV capsid precursor protein at least comprises the P1 and 2A proteins (also referred to herein as P1-2A capsid precursor).

A “virus-like particle” (VLP), which may also be referred to in the art as “empty capsid”, is an entity which comprises the protein shell of a virus but lacks the RNA or DNA genome. A VLP should be antigenic and immunogenic in the same way as the wild-type virus because it retains the same structural epitopes, but it should produce no infection, due to the lack of the virus genome. An FMDV VLP is typically formed from the P1-2A capsid precursor. As described above, the 2A protease cleaves itself at its C terminus to release P1-2A from P2. Processing of the P1-2A capsid precursor is affected by the 3C protease to produce 2A and the capsid proteins VP0, VP3 and VP1. The VLP is formed by self-assembly from these capsid proteins.

VLPs may also be produced in the baculovirus expression system of the present invention using a modified 3C protease that is less toxic to the insect cells (Porta et al. (2013) J Virol Methods). Intermediate and non-toxic activity of the 3C enzyme in a P1-2A-3C expression cassette allows recombinant expression and processing of the P1-2A precursor into the structural proteins, VP0, VP1, and VP3, which assemble into VLPs. The production of VLPs may be investigated or verified using techniques known in the art such as sucrose density centrifugation or electron microscopy (Abrahams et al (1995)). Monoclonal antibodies may be used specific for conformational epitopes on the wild-type virus in order to investigate whether the structure and antigenicity of the empty capsid is retained.

The term “vaccine” as used herein refers to a preparation which, when administered to a subject, induces or stimulates a protective immune response. A vaccine can render an organism immune to a particular disease.

To “protect an animal against an infection with FMDV” means aiding in preventing, ameliorating or curing a pathogenic infection with FMDV, or aiding in preventing, ameliorating or curing a disorder arising from that infection, for example to prevent or reduce one or more clinical signs resulting from a post treatment (i.e. post vaccination) infection with FMDV.

The term “prevention” or “preventing” is intended to refer to averting, delaying, impeding or hindering the FMDV infection by a prophylactic treatment. The vaccine may, for example, prevent or reduce the likelihood of an infectious FMDV entering a cell.

### Baculovirus Expression Vector

The baculovirus expression vector of the first aspect of the present invention is capable of recombinantly expressing an FMDV capsid precursor protein under control of a promoter. The expression vector comprises the two translational enhancers Syn21 and p10UTR, which enhance the recombinant expression of the FMDV capsid precursor protein.

The translational enhancer “Syn21” is an AT-rich synthetic sequence of 21 nucleotides (nt) made by combining the Cavener consensus sequence with elements from the Malacosoma neustria nucleopolyhedrovirus (MnNPV) polyhedrin gene as described in “B. D. Pfeiffer et al, PNAS (2012), Vol. 109(17), p. 6626-6631”. The nucleic acid sequence of the Syn21 translational enhancer may have a nucleic acid sequence corresponding to the nucleic acid sequence 5′-AAC TTA AAA AAA AAA ATC AAA-3′ (SEQ ID NO.1). In this invention, the translational enhancer Syn21 is located within the 5′ untranslated region (UTR) of the nucleic acid sequence encoding the FMDV capsid precursor protein. In a preferred embodiment, the Syn21 sequence is located in direct proximity 5′ to the ATG start codon of the nucleic acid sequence encoding the FMDV capsid precursor protein.

The translational enhancer “P10UTR” is located within the 3′ UTR of the nucleic acid sequence encoding the FMDV capsid precursor protein. The term “P10UTR” as used herein relates to the 3′ UTR from the AcNPV p10 gene as described in “Y. Liu et al., Biotechnol. Lett. (2015), Vol. 37, p. 1765-1771”. Preferably, the P10UTR has a nucleic acid sequence corresponding to the nucleic acid sequence of SEQ ID NO. 2.

Encompassed by the terms “Syn21” and “P10UTR” are nucleic acid sequences corresponding to those of SEQ ID NO. 1 and 2, but including conservative modifications, such as mutation and/or natural variation, of one or more nucleic acids. A modification may be a deletion or addition of one or more nucleotides or a replacement of one or more nucleotides by one or more other nucleotides. A conservative modification is typically a modification that does not substantially alter the function of the sequence as translational enhancer, i.e. the modified sequence is still capable of enhancing expression under control of the promotor of the expression cassette.

The FMDV capsid precursor protein is recombinantly expressed under the control of a suitable promoter. The promoter is not particularly limited but may be any promoter capable for the recombinant expression of the FMDV capsid precursor protein in a baculovirus expression system. Preferred promoters for use in the baculovirus expression system of the present invention are the polyhedrin (polh) promoter (described in: Ayres M. D. et al., Virology (1994), Vol. 2020, p. 586-605) and the p10 promoter (described in: Knebel D. et al., EMBO J. (1985) Vol. 4(5), 1301-1306) of AcNPV. Another preferred promoter is the promoter of the orf46 viral gene of S. exigua nucleopolyhedrovirus (SeNPV) (described in M. Martinez-Solis et al., PeerJ (2016), DOI 0.2183).

Baculovirus expression vectors for use in baculovirus expression systems for the recombinant expression of proteins are commercially available and are extensively used in the art for the production of proteins and virus-like particles. The systems may encompass, for example, one or more transfer plasmids used to transform cells, such as E. coli cells or insect cells, in which the baculovirus expression vector is propagated. Commercially available baculovirus expression vectors include, but are not limited to, Top-Bac® vector (ALGENEX, Spain), pFastBac® vector (Thermo Fisher Scientific, Germany), flashBAC® vector (Oxford Expression Technologies Ltd, UK) and BestBac® vector (EXPRESSION SYSTEMS, CA).

The baculovirus expression vector of the present invention contains an expression cassette comprising the nucleic acid sequence encoding the FMDV capsid precursor protein, which is expressed in the host cell under control of a functional promoter, and including the Syn21 and P10UTR translational enhancers.

An expression cassette for the recombinant expression of a FMDV capsid precursor protein in the present invention thus typically has the schematic structure shown in FIG. 1B.

This structure may comprise additional elements, in particular other cis-acting elements, such as additional translational enhancers, which can be used in combination with the translational enhancers Syn21 and p10UTR in the baculovirus expression vector of the present invention.

The nucleic acid sequence encoding the FMDV capsid precursor protein is not particular limited and may be of any FMDV serotype, such as of serotypes A, O, Asia1, SAT1, SAT2, SAT3 and C. In a particularly preferred embodiment, the FMDV capsid precursor protein is from the A or O serotype.

In the baculovirus expression vector of the present invention, the capsid precursor protein typically comprises at least the capsid precursor P1. More preferably, the capsid precursor protein comprises the capsid precursor P1 and the 2A peptide.

In a further preferred embodiment, the baculovirus expression vector of the present invention further comprises a nucleic acid sequence encoding a protease capable of cleaving an FMDV capsid precursor protein. The protease may be any protease capable of cleaving the FMDV capsid precursor protein as a step in the production and assembly of capsids in order to produce FMDV empty capsids. As mentioned above, for FMDV, proteolytic processing of the precursor P1 into VP0 (VP2 plus VP4), VP3 and VP1 occurs by means of the viral 3C protease or its precursor 3CD. Hence, the protease is preferably the 3C protease of FMDV. The sequence of FMDV wild-type 3C protease from an FMDV type A strain is described in the art and is disclosed in WO 2011/048353, which is hereby incorporated by reference in its entirety. The 3C protease may also be a functional derivative including one or more mutations, which reduce its proteolytic activity, for example a mutation at Cysteine 142.

In a further preferred embodiment, the baculovirus expression vector of the first aspect may be a nucleotide sequence which comprises (i) the nucleic acid sequence of the FMDV capsid precursor, (ii) the translational enhancer Syn21, (iii) the translational enhancer P10UTR, and (iv) the nucleic acid sequence of the protease. The nucleotide sequences of (i) the FMDV capsid precursor and (iv) the protease are preferably arranged in a contiguous manner. There may be a nucleic acid sequence between nucleic acid sequences (i) and (iv). A control element, such as a control element as described in WO 2011/048353, which is hereby incorporated by reference in its entirety, may be present in that sequence, such that it controls expression of the protease but does not control or affect expression of the capsid precursor protein.

The capsid precursor protein may be cleavable by the protease to form (part of an) empty capsid. The precursor protein may comprise all proteins necessary to form an empty capsid.

The capsid precursor protein may be P1, which is cleaved by the 3C protease into VP0, VP3 and VP1. Alternatively, the capsid precursor protein may be P1-2A. The 2A peptide cleaves itself at its C terminus to release P1-2A from any downstream protein sequence. Most preferably, the baculovirus expression system expresses a P1-2A-3C cassette, i.e. it simultaneously expresses the coding regions for the proteins P1, 2A and 3C. Expression of the 3C enzyme in a P1-2A-3C cassette allows expression and processing of the P1-2A precursor into the structural proteins which assemble into VLPs. The capsid precursor protein and the protease may be expressed under control of individual promotors or under control of the same promoter. For example, the capsid precursor protein may be expressed under control of a first promoter as described herein and wherein gene expression is regulated by the Syn21 and p10UTR translational enhancers, and the protease is expressed under control of a separate (second) promoter, which may be different from the first promoter.

Cleavage of the capsid precursor protein or VLP may be analysed using techniques known in the art. For example, extracts from baculovirus-infected host cells may be separated by gel-electrophoresis and the separated proteins transferred onto a nitrocellulose membrane for Western blotting. Western blotting with protein-specific antibodies should reveal the degree of protease-mediated cleavage. For example, for FMDV, the unprocessed capsid precursor protein (P1-2A) would appear as a band of around 81 kDa, and cleavage may produce VP3-1 (˜47 kDa), VP0 (˜33 kDa), VP2 (˜22 kDa), VP3 (˜24 kDa) and/or VP1 (˜24 kDa).

### Host Cell

In a second aspect, the invention provides a host cell comprising the baculovirus expression vector according to the first aspect of the invention. In a further embodiment, the host cell is capable of producing capsid precursor proteins, and preferably is capable of producing FMDV VLPs.

The host cell may, for example, be a bacterial cell, an insect cell, plant cell or a mammalian cell. Preferably, the host cell is an insect cell, such as a Sf9 cell (a clonal isolate of Spodoptera frugiperda Sf21 cells), or a Tni cell (ovarian cells isolated from Trichoplusia ni). Most preferably, the host cell is a Tni cell, or a Tni-derived cell line, such as a Tnao38 cell.

### Method of Producing Virus-Like Particles

In a third aspect, the invention provides a method of producing FMDV capsid precursor protein. Alternatively, the invention provides a method of producing FMDV VLPs. The method according to the third aspect comprises the steps of:
(i) infecting a host cell according to the second aspect with the baculovirus expression vector according to the first aspect, and
(iia) harvesting FMDV capsid precursor protein produced by the host cell, or
(iib) harvesting FMDV VLPs produced by the host cell.

The method thus includes the culturing of the host cell under conditions suitable for the host cell to express the capsid precursor protein from the baculovirus expression vector in order to produce capsid precursor protein. In case the baculovirus expression construct further expresses a protease capable of cleavage of the capsid precursor protein, as described above, FMDV VLPs may be produced by the host cell. If the VLPs are released by the host cell, they may be harvested from the cell culture medium. If the empty virus capsids are retained inside the host cell, they may be harvested by, for example,
(i) lysis of the host cells (for example by freeze-thawing); and optionally
(ii) concentration (e.g. by PEG-precipitation), and/or
(iii) purification.

### Vaccines and Production Thereof

The present invention further relates to the production of FMDV VLPs, which are used in the production of a vaccine.

The present invention thus also provides a method for the production of a vaccine, which comprises the step of producing FMDV VLPs by a method according to the third aspect and incorporating the FMDV VLPs in a vaccine, such as by the addition of a pharmaceutically acceptable carrier.

Pharmaceutically acceptable carriers are well-known in the art. Merely as an example; such a carrier can be as simple as sterile water or a buffer solution such as PBS. The vaccine may comprise a single carrier or a combination of two or more carriers. The vaccine may also comprise one or more pharmaceutically acceptable diluents, adjuvants and/or excipients. The vaccine may also comprise, or be capable of expressing, another active agent, for example one which may stimulate early protection prior to the vaccinating entity-induced adaptive immune response. The agent may be an antiviral agent, such as type I interferon. Alternatively, or in addition, the agent may be granulocyte-macrophage colony-stimulating factor (GM-CSF).

The vaccine may be used therapeutically, to treat an existing FMDV infection (especially in herds or regions where the virus is endemic), but preferably is used prophylactically, to block or reduce the likelihood of FMDV infection and/or prevent or reduce the likelihood of spreading the disease.

Many commercially available FMD vaccines are multivalent to provide protection against the different FMD serotypes. By the same token, the vaccine of the present invention may comprise a plurality of vaccinating entities, each directed at a different serotype and/or different subtypes within a given serotype.

### Treatment

The present invention also provides a method of protecting a subject against an infection with FMDV by administration of an effective amount of a vaccine of the present invention.

For FMD the subject may be a cloven-hoofed animal. FMD susceptible animals include cattle, sheep, pigs, and goats among farm stock, as well as camelids (camels, llamas, alpacas, guanaco and vicuna). Some wild animals such as hedgehogs, coypu, and any wild cloven-footed animals such as deer and zoo animals including elephants are also susceptible to FMD.

### Administration

The present invention contemplates at least one administration to an animal of an efficient amount of the vaccine according to the invention. A vaccine can be administered in any art-known method, including any local or systemic method of administration. Administration can be performed e.g. by administering the antigens into muscle tissue (intramuscular, IM), into the dermis (intradermal, ID), underneath the skin (subcutaneous, SC), underneath the mucosa (submucosal, SM), in the veins (intravenous, IV), into the body cavity (intraperitoneal, IP), orally, anally etc. For the current vaccine IM, ID and SC administration are preferred.

### FIGURES

FIG. 1 schematically shows DNA structures for use according to the invention

FIG. 2 represent in various ways yields when using the invention

FIG. 3 shows a Western Blot to indicate yield when using the invention

### EXAMPLES

The invention will be further described by way of the following non-limiting examples, which are meant to serve to assist one of ordinary skill in the art in carrying out the invention.

### “Opt1” Baculovirus Construct

The transcription of the gene-of-interest (GOI) in the commercially available standard baculovirus transfer vector, pFastBac® (Thermo Fisher Scientific, Germany) is driven by the polyhedrin (polh) promoter. The resulting mRNA contains a SV40 3′UTR.

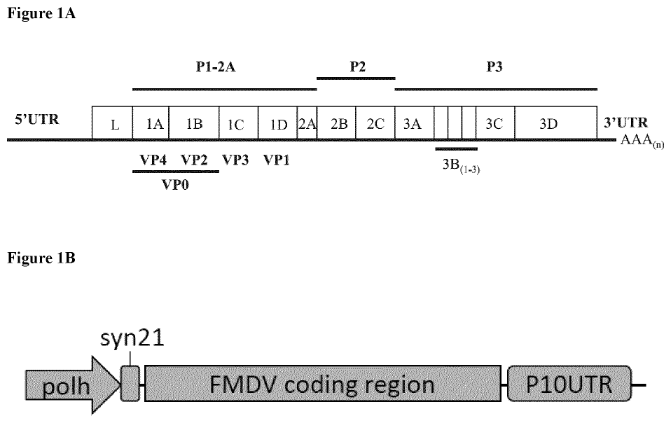

To investigate whether the translation of this mRNA can be improved, the SV40 3′UTR was replaced by the 3′-UTR from the Autographa californica nucleopolyhedrovirus (AcNPV) p10 gene (P10UTR). In addition, the Syn21 translational enhancer was placed in the 5′UTR of the mRNA and just in front of the open reading frame (ORF) encoding the FMDV capsid precursor protein. The resulting modification of the expression cassette was designated “Opt1” (FIG. 1B). Expression of the FMDV capsid precursor protein from the standard expression cassette was compared to expression of the FMDV capsid precursor protein from the “Opt1” expression cassette.

Cloning of the expression cassette O/TUR/5/09 VP2-S93F used in Example 1 and of the expression cassette A/IRN/7/13 VP2-H93F used in Example 2 was performed by standard cloning procedures well known in the art. The nucleotide sequence of the expression cassette of O/TUR/5/09 VP2-S93F is according to the nucleic acid sequence of SEQ ID NO. 3. The nucleotide sequence of the expression cassette A/IRN/7/13 VP2-H93F is according to the nucleic acid sequence of SEQ ID NO. 4.

### Example 1

In this Example, the expression level of an FMDV capsid precursor protein of an O serotype from the Opt1 expression cassette is compared to the expression from a standard expression cassette for obtaining high yields.

Erlenmeyer flasks with 100 ml containing 3.2×10⁵ cells/ml of Tni cells were infected at MOI=1 with recombinant baculoviruses containing the P1-2A-3Cpro expression cassette based on O/TUR/5/09 VP2-S93F. The culture was harvested at 4 dpi and the cells were collected by centrifugation and subsequently sonicated in Tris-KCl pH8.0 buffer at one-tenth of the original culture volume.

Samples were analyzed by Western blotting using the anti-VP0 monoclonal antibody (Loureiro et al., 2018, https://wellcomeopenresearch.org/articles/3-88). Visual inspection of the Western blot suggests that the Opt1 version of the baculovirus vector performs slightly better than the standard one in terms of yield of FMDV-related proteins.

To quantify the differences between the 2 baculovirus constructs, ELISA was performed using monoclonal antibody INT-FMA-01-08 which detects both intact capsids (75S/146S) and pentameric building blocks of the capsids (12S). For this, serially diluted samples were incubated for 1 h at 37° C. on microtiter plates coated overnight at 4° C. with antibody. After removing the samples and three washes with PBS-Tween, a fixed amount of biotinylated INT-FMA-01-08 antibody was added to plates and incubated for 1 h at 37° C. The biotinylated antibody was removed and plates were washed three times with PBS-Tween, after which peroxidase-conjugated streptavidin was added to the plates followed by chromophoric detection.

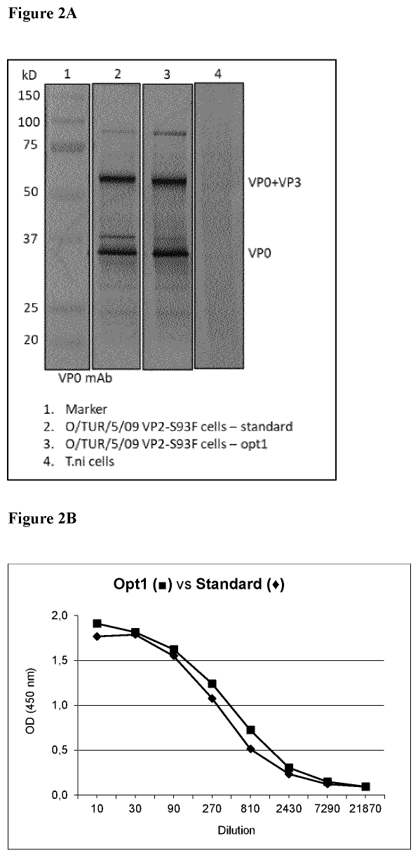

Results of Western blotting are shown in FIG. 2A. Results of ELISA are shown in FIG. 2B. The ELISA shows an increase in the expression of the FMDV-related proteins of the O serotype of 1.4-fold for the Opt1 expression cassette compared to the standard expression cassette. This may not seem substantial but given the fact that a production run typically takes 5 days, this means that the same amount of antigen can be made in 10 days (2 runs) using the Opt1 expression cassette, compared to about 15 days (3 runs) when using the other expression cassette.

### Example 2

In this Example, the expression level of an FMDV capsid precursor protein of an A serotype from the Opt1 expression cassette is compared to the expression from the standard expression cassette.

Infection of cells and cell culture was performed as described in Example 1, except that the expression cassette based on A/IRN/7/13 VP2-H93F was used.

Samples were analyzed by Western blotting as described in Example 1. Visual inspection of the Western blot suggests that the Opt1 version of the baculovirus vector performs better than the standard one in terms of yield of FMDV-related proteins.

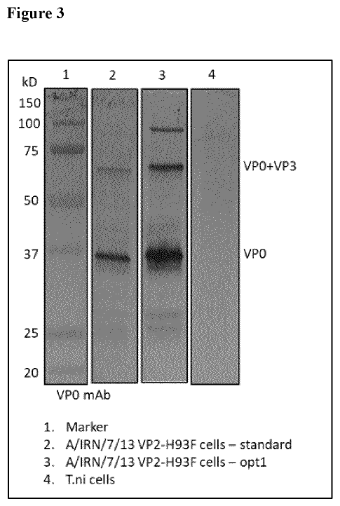

Results of Western blotting are shown in FIG. 3 Based on band intensities, it is estimated that the increase in the expression of the FMDV-related proteins of the A serotype is 3-fold for the Opt1 expression cassette compared to the standard expression cassette.

### Example 3

In this Example, the expression level resulting from the Syn21 and P10UTR translational enhancers in the Opt1 expression cassette was compared with a commercial system (TopBac®, Algenex). In the TopBac® expression vector, expression is achieved under the control of the polyhedron (polh) promoter, and a homologous repeated (hr) transcription enhancer sequence operatively cis-linked to p10 chimeric promoters. The TopBac ° expression vector was described to achieve an up to 4-fold increase in the production yield of a recombinant model protein (green fluorescent protein, GFP) with respect to a standard baculovirus vector. (Löpez-Vidal, J. et al, PLoS ONE 10(10): e0140039).

Infection of cells and cell culture was performed as described in Example 1.

Cell and supernatant were analyzed by Western blotting as described in Example 1. Visual inspection of the Western blot suggests that the Opt1 version of the baculovirus vector outperforms both the commercial TopBac ° vector in terms of yield of FMDV-related proteins.

To better quantify the differences between the 2 constructs, ELISA was performed as described in Example 1.

The ELISA data confirmed the Western blot results in that the Opt1 expression vector provided overall higher protein yields than the TopBac® vector. In fact, the TopBac® vector, although successful for many other proteins like GFP, does not achieve the high level of expression of the FMDV P1-2A-3Cpro cassette of the Opt1 expression vector (see Table 1 below).

TABLE 1
ELISA results showing expression level resulting from
the Opt1 construct compared to a commercial system.
Expression levels obtained with Opt1 were set at 100%.

| | ELISA (%) | |
|---|---|---|
| Baculovirus | | cell culture 30 |
| expression vector | cells | medium |
| Opt1 | 100 | 100 |
| Top-Bac | 27 | 20 |

### CONCLUSIONS

For two FMDV strains belonging to two different serotypes, O and A, an increase in FMDV protein expression levels was obtained by using the Opt1 baculovirus construct comprising the Syn21 and P10UTR translational enhancers compared to a standard expression system. Surprisingly, an increase in expression level by using the Opt1 baculovirus expression vector was even achieved compared to a commercial system containing other translational enhancers, and which is described in the art to achieve an increase in expression level and protein yield.

1. A baculovirus expression vector capable of recombinantly expressing a Foot and mouth disease virus (FMDV) capsid precursor protein under control of a promoter, the expression vector comprising:
(i) a nucleic acid sequence encoding the FMDV capsid precursor protein,
(ii) a translational enhancer Syn21 located within the 5′ untranslated region (UTR) of the nucleic acid sequence (i) encoding the FMDV capsid precursor protein, and
(iii) a translational enhancer P10UTR, located within the 3′UTR of the nucleic acid sequence (i) encoding the FMDV capsid precursor protein.

2. The baculovirus expression vector according to claim 1,
wherein the translational enhancer Syn21 has a nucleic acid sequence corresponding to the nucleic acid sequence of SEQ ID NO. 1.

3. The baculovirus expression vector according to claim 1,
wherein the translational enhancer P10UTR has a nucleic acid sequence corresponding to the nucleic acid sequence of SEQ ID NO. 2.

4. The baculovirus expression vector according to claim 1, wherein the FMDV is of the A serotype.

5. The baculovirus expression vector according to claim 1, wherein the FMDV is of the O serotype.

6. The baculovirus expression vector according to claim 1, wherein the capsid precursor protein comprises the capsid precursor P1.

7. The baculovirus expression vector according to claim 1, wherein the vector further comprises:
(iv) a nucleic acid sequence encoding a protease capable of cleaving the capsid precursor protein into one or more capsid proteins.

8. The baculovirus expression vector according to claim 7, wherein the capsid precursor protein comprises the capsid precursor P1 and the 2A peptide and the protease is 3C.

9. A host cell comprising the baculovirus expression vector according to claim 1.

10. The host cell according to claim 9, which is an insect cell.

11. A method of producing FMDV capsid precursor protein, the method comprising the steps of:
(i) infecting a host cell with the baculovirus expression vector according to claim 1, and
(ii) harvesting FMDV capsid precursor protein produced by the host cell.

12. A method of producing FMDV virus-like particles (VLPs), the method comprising the steps of:
(i) infecting a host cell with the baculovirus expression vector according to claim 7, and
(ii) harvesting FMDV VLPs produced by the host cell.

13-14. (canceled)

15. A method of producing a vaccine, which comprises the steps of:
(i) producing FMDV VLPs by the method according to claim 12 and
(ii) incorporating the FMDV VLPs into a vaccine by addition of a pharmaceutically acceptable carrier.

16. A method of protecting a subject against an infection with FMDV, which comprises the step of expressing an FMDV capsid precursor protein from the baculovirus expression vector according to claim 7 in a host cell to produce a VLP, incorporating the VLP into a vaccine by addition of a pharmaceutically acceptable carrier and administering the vaccine to the subject.

17. (canceled)


SEQUENCE LISTING (4 sequences)

SEQ ID NO: 1 - DNA, 21 bp, Artificial Sequence
  Syn21 translational enhancer
aacttaaaaaaaaaaatcaaa

SEQ ID NO: 2 - DNA, 666 bp, Artificial Sequence
  P10UTR translational enhancer
atgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccagtattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaac

SEQ ID NO: 3 - DNA, 3781 bp, Artificial Sequence
  expression construct syn21-O/TUR/5/09-P10UTR
aacttaaaaaaaaaaatcaaaggatccggtaaccaaatatgggggcgggtcaatcgtcaccagcaacggggtctcagaatcaaagcgggaacacggggtctattatcaacaactactacatgcagcaataccaaaacagcatggacacccaactgggcgacaacgctatctctggcggttcaaacgagggctcaactgacaccaccagcaaccacacgaccaatacccaaaacaatgactggttctctaagctcgccagcagcgctttcagcggcctgttcggtgctcttcttgcagacaagaaaaccgaggaaactacgctgctggaggaccgtatcctgaccactaggaacggccacacgaccagcactacgcaaagcagcgttggcgtgacctacggttatgctaccactgaagacttcgtttccggtcccaacaccagcggcctggagactcgtgttgtgcaggctgaacgtttcttcaagacccatctgttcgactgggttactagcgatagcttcggacgttgccacctgctggagctgccaactgaccataaaggagtgtacggcttcctgaccgattcctacgcctatatgcgtaacggctgggacgtcgaagtaaccgctgttggcaaccaattcaatggagggtgcctactagttgctatggtgcccgagctgtgtagcatcaacaagcgtgaactgtaccaattaactctgttcccgcaccagttcatcaacccacgtaccaatatgactgctcatattaccgttcccttcgttggcgtgaaccgttacgaccaatataaggtgcacaaaccctggaccctggttgttatggttgtggcccccctcaccgtgaacactgaaggcgctccccaaatcaaggtctacgcgaacattgctcccaccaacgtccatgtagccggagagttccccagcaaagaaggcatcttccctgttgcttgcagcgacggatacggcggtctggtgacgaccgaccccaagaccgctgaccccgcatacggtaaagttttcaacccccctaggaacatgctgcccggacgtttcactaacttccttgacgtggccgaggcttgccctaccttccttcacttcgaaggcgatgtcccctacgtaactacgaagactgacagcgatcgtatcctcgctcaattcgaccttagcctcgctgccaaacacatgagcaacaccttcctggctggacttgcccaatactatacccagtacagcggcactatcaacctgcatttcatgttcaccggtcccactgacgcgaaggctcgttacatgatcgcctatgcaccgcccggtatggagccccctaaaacccccgaagctgctgctcactgcatccatgctgagtgggacaccggcctgaactctaagttcactttctcaattccctaccttagcgctgctgactacgcatataccgcttctgatactgctgaaaccactaacgtccaaggctgggtttgtctgttccagatcacccacggaaaggctgacggcgacgctctggttgtactagctagcgccggcaaagacttcgagctgcgcttacccgttgacgctaggacccaaacgaccagcgctggcgaatcagctgaccccgtgaccgctactgtcgagaactacggaggggaaacccaagtccagcgtcgtcaacatactgacgtttctttcatcctggaccgtttcgttaaagtgacccccaaagaccaaatcaacgttctggacctgatgcagaccccagctcacactctggtgggcgctctgttacgtaccgctacttactatttcgctgacctggaggtcgctgtaaagcatgaaggtaatctgacctgggttcccaacggcgctcccgagactgctctggacaacactacgaatccgaccgcttaccacaaggcccctctgacccgtctcgccctaccgtacactgctccacatcgcgtgcttgccaccgcatacaacggcaattgcaagtacggtgaatctcacaccactaacgttcgtggagacctgcaagtgcttgctcagaaagcggctcgcaccctgcccacttcattcaactacggcgccatcaaggcaacccgtgttactgagctgctttacaggatgaaacgtgctgaaacctactgtccccgtcccctcctagccatccacccctcagaggctcgccataagcaaaaaattgttgctcctgtgaagcaattattaaatttcgacctactgaagttagctggggacgttgaatcgaacccgggacccagcggccgcggaccttttttagggaagatctggccttcctacaagggaaggccagggaattttcttacgagggacctgtgaagaagcctgtcgctttgaaagtgaaagctaagaacttgattgtcactgagagtggagccccaccgaccgacttgcaaaagatggtcatgggcaacaccaagcctgttgagcttatcctcgacgggaagacggtggccatttgttgtgctaccggagtgtttggcactgcgtacctcgtgcctcgtcatctttttgcagaaaaatatgacaagatcatgctggacggcagagccatgacagacagtgactacagagtgtttgaattcgagattaaagtaaaaggacaggacatgctctcagacgctgcgctcatggtactccaccgtgggaatcgcgtgagagacatcacgaaacactttcgtgacacagcaagaatgaagaaaggcacccctgttgtcggagtgatcaacaatgccgacgttgggagactgatcttctctggtgaggccttaacctacaaggacattgtagtgactatggatggagacaccatgcctggcctgtttgcctacaaagccgccaccaaggctggctactgtgggggagccgttcttgctaaggacggagctgacacattcatcgttggtacccactccgcaggcggcaatggagttggatactgctcatgcgtctcgaggtccatgttgctgaaaatgaaggcgcacatcgaccccgaaccacaccacgagtaatctagaggatcccctcaggaagcttgtcgagaagtactagagatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccagtattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaac

SEQ ID NO: 4 - DNA, 3784 bp, Artificial Sequence
  expression construct syn21-A/IRN/7/13-P10UTR
aacttaaaaaaaaaaatcaaaggatccggtaaccaaatatgggagcgggacaatcgtcgcctgccacggggagccaaaaccaatctgggaacactggaagcatcatcaataactactatatgcaacaataccaaaacagcatggacacccaactgggcgacaacgctatcagcggcggttctaacgagggatctaccgacaccacttcaactcacacgaccaacacccaaaacaatgactggttcagcaagctcgctagctctgccttcagcggcctgttcggcgctctgcttgcggacaagaaaaccgaggaaactacgctgctggaggaccgtatcctcaccactcgcaacggccacacgaccagcactacgcaaagcagcgtcggagtaacttacgggtattctaccggagaagaccatgtttcgggaccaaacaccagcggcctggagacccgtgttgtgcaggctgaacgtttcttcaagaaacacctgttcgactggaccactgataaggctttcggccatcttgagaaacttgaactccccaccgagcacaagggagtttacgggttcctggtggactctttcgcttacatgcgtaacggctgggacgtcgaagtgaccgccgttggcaaccaattcaatggagggtgcctactagttgctatggtgcccgagtggaaggagttcacgacccgtgaaaaataccaactaaccttattcccgcaccagttcatcaaccctcgtaccaatatgactgctcatattaccgttccctaccttggcgttaaccgttacgaccaatataagcagcacaaaccctggaccctggttgttatggttgtgagccctcttactaccagcaacatcggcgcttcacaaatcaaggtttacgctaacatcgcccccaccttcgtccatgtagctggcgagctgccctcaaaagaaggaatcgttccagtggcttgcagcgacggatacggcggtctggttactactgaccccaagacagctgaccccgtctacggcatggtatataacccccctcgtactaattaccccggtcgcttcaccaaccttctcgacgtggctgaggcttgccccaccttcctgtgtttcgacggaggcaagccctacgttgaaaccaggactgacgctcaacgtctattagccaagttcgatgtgtcactcgctgccaaacacatgtcgaacacctacctgagcggaatcgctcaatactatactcagtacagcggcaccatcaaccttcatttcatgttcaccggttcaactgactcgaaggcacgttacatggttgcctatatcccgccaggaatggacaccccccctgatactcctgagaaggcagcgcactgcatccatgctgaatgggacactggtctcaactctaaattcaccttctcaatcccctacgttagcgctgccgactacgcatatactgccagcgatgaggcagaagcgaccaacgttcaaggatgggtgtgtatctaccaaattactcacggcaaggctgagcaagacaccctggttgttagcgccagcgctggcaaggacttcgaactgaggcttcccatcgatccccgtgctcaaacgactactgcaggagagtcagccgaccccgtcactaccactgttgagaactacggcggtgaaactcaagttcagcgtcgtcaccataccgacgtgggattcatcatggatcgtttcgtgaagatcagcccggtcggcccaactcacgtgatcgacctcatgcaaacccaccaacacgctctggttggcgctctgcttcgtgctgcgacctactatttcagcgaccttgagatcgttgtgcgtcacgaaggtcatctcacctgggtgcccaacggcgctcccgtgggcgctctggttaacacctctaatcctactgcttactgcaaggagcccttcactcgtctcgctctaccgtacaccgccccacaccgcgttcttgctaccgtgtacaacggcgttagcaaatacagcaccactggaggcgacaggcgtggcgacctgggtagccttgctgcccgtgttgcagcgcaccttcccagcagcttcaacttcggcgccatcaaggcaaccaacattcatgagctgctggttcgtatgaaaagggctgaactctactgtccccgtcccttactggccgtcgaggttagcagccaagaccgccacaagcagaaaatcattgcacccgcgaagcaattactgaacttcgacctactgaaattagcgggagacgttgaatcgaaccccggacccagcggccgcggaccttttttagggaagatctggccttcctacaagggaaggccagggaattttcttacgagggacctgtgaagaagcctgtcgctttgaaagtgaaagctaagaacttgattgtcactgagagtggagccccaccgaccgacttgcaaaagatggtcatgggcaacaccaagcctgttgagcttatcctcgacgggaagacggtggccatttgttgtgctaccggagtgtttggcactgcgtacctcgtgcctcgtcatctttttgcagaaaaatatgacaagatcatgctggacggcagagccatgacagacagtgactacagagtgtttgaattcgagattaaagtaaaaggacaggacatgctctcagacgctgcgctcatggtactccaccgtgggaatcgcgtgagagacatcacgaaacactttcgtgacacagcaagaatgaagaaaggcacccctgttgtcggagtgatcaacaatgccgacgttgggagactgatcttctctggtgaggccttaacctacaaggacattgtagtgactatggatggagacaccatgcctggcctgtttgcctacaaagccgccaccaaggctggctactgtgggggagccgttcttgctaaggacggagctgacacattcatcgttggtacccactccgcaggcggcaatggagttggatactgctcatgcgtctcgaggtccatgttgctgaaaatgaaggcgcacatcgaccccgaaccacaccacgagtaatctagaggatcccctcaggaagcttgtcgagaagtactagagatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccagtattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaac